(19) United States
(12) Patent Application Publication
(10) Pub. No.: US 2025/0363773 A1
(43) Pub. Date: Nov. 27, 2025

(54) TECHNOLOGIES FOR AUTOMATED ORTHOPAEDIC SURGICAL TRAY INSPECTION

(71) Applicant: JOHNSON & JOHNSON SERVICES, INC., NEW BRUNSWICK, NJ (US)

(72) Inventors: Nastaran Emaminejad, Rancho Mission Viejo, CA (US); Suchitra Ganapathi, Redmond, WA (US); Sandhya Kamineni, Basking Ridge, NJ (US); Reyhaneh Nosrati, Boston, MA (US); Saman Sarraf, San Jose, CA (US); Yuqing Sun, Bellevue, WA (US)

(21) Appl. No.: 19/216,114

(22) Filed: May 22, 2025

Related U.S. Application Data
(60) Provisional application No. 63/651,139, filed on May 23, 2024.

(51) Int. Cl. G06V 10/75; A61B 50/33; G06T 7/00; G06T 7/33; G06T 7/73; G06V 10/774; G06V 10/82

(52) U.S. Cl. CPC G06V 10/751; G06T 7/0014; G06T 7/337; G06T 7/74; G06V 10/774; G06V 10/82; A61B 50/33; G06T 2207/20081; G06T 2207/20084; G06V 2201/034

(57) ABSTRACT

A system for automated surgical instrument tray inspection includes a user device in communication with an inspection device. The user device captures a test image of an instrument tray being inspected and sends the image to the inspection device. The inspection device determines a unique tray identifier of the instrument tray. The inspection device generates object predictions from the test image with a trained object recognition model, post-processes the object predictions with non-max suppression based on a predetermined tray configuration associated with the tray identifier, and determines whether the object predictions match a predetermined tray layout associated with the tray identifier. The user device displays a user interface indicating whether the object predictions match the predetermined tray layout. Other embodiments are described and claimed.

### CROSS-REFERENCE TO RELATED APPLICATION

This application claims the benefit of and priority to U.S. Patent Application No. 63/651,139, entitled “AUTOMATED SOLUTION FOR ORTHOPEDIC SURGICAL TRAY INSPECTION,” which was filed on May 23, 2024, and which is incorporated herein by reference in its entirety.

### TECHNICAL FIELD

The present disclosure relates generally to automated visual inspection tools and, more specifically, to automated visual inspection tools in manufacturing and supply chain processes for the delivery of orthopaedic components.

### BACKGROUND

Application of automated visual inspection tools in manufacturing and supply chain processes in industry can significantly enhance efficiency, improve reliability, and increase quality. In the past years, computer vision has received attention in building automated inspection tools for industry. Computer vision approaches can help in reducing errors that can occur in manual techniques. Furthermore, computer vision-based technologies can allow real-time approaches that can help in avoiding delays and can be made highly scalable for various production environments. These capabilities enable digitalization and automation for various industrial use cases.

In the medical devices industry, a use case for automated inspection tools is in quality control via inspecting for defects, missing or misplaced elements of a medical product such as surgical trays. Johnson & Johnson MedTech offers an extensive range of orthopedic joint reconstruction trays, each comprising a collection of distinct but highly similar components placed in designated slots. The current practice for tray inspection involves manual examination of all trays before shipment or upon their return to ensure the presence and correct placement of components.

In recent years, deep learning models like convolutional neural networks (CNN) have greatly impacted the computer vision field including its application in industrial manufacturing and supply chain. Deep learning offers high accuracy, speed and adaptability compared to traditional computer vision methods. Algorithms such as Region Based Convolutional Neural Network (“RCNN”) families of models and You Only Look Once (“YOLO”) models are two of the most popular object detection algorithms and have shown significant improvements in building intelligent inspection tools for various industrial use cases such as detection of small hardware components and parts like screws.

The initial version of the RCNN model was built through two main stages of region proposal followed by application of a CNN for classification. A faster-RCNN model was then introduced and was improved via implementation of a cost-free Region Proposal Network (RPN) for the first stage and a Feature Pyramid Network (FPN) for the classification stage. RPN shares full-image convolutional features with the detection network and FPN as a top-down architecture with lateral connections builds high-level feature maps.

YOLO is an object detection model that performs the detection task in a single forward pass at high-speed making it suitable for applications that require low-latency processing. Since YOLO was first proposed, the model architecture has received incremental improvements throughout the years, while the most recent versions YOLOv7 and YOLOv8 outperform the earlier versions in mAP and quick inference. The YOLO framework consists of three main components: the backbone, head, and neck. YOLOv7 incorporates the Extended Efficient Layer Aggregation Network (E-ELAN) as its computational block within the backbone. The neck collects feature maps extracted by the backbone and creates feature pyramids. Finally, the head consists of output layers that have final detections. YOLOv8 further refines this concept by introducing novel architectural enhancements and optimization strategies, aiming to achieve even higher accuracy and efficiency.

Several deep learning methods have been developed and used for object detection across a wide range of applications such as autonomous driving, electronics, and supply chain processes, e.g., defect detection and visual inspection.

Among industry use cases, an approach based on Faster-RCNN was built for defect detection of printed circuit boards (PCB) which achieved mAP of up to 95.6%. The study was trained on 1750 images of circuit boards from an electronics factory and utilized a deeper ResNet50 and FPN backbone for feature extraction and replaced original RPN with a multi-scale RPN for more accurate region proposal that improved defect detection performance. Another study based on Faster-RCNN for defect detection, used multi-scale feature maps and a top-down fusion of low- and high-resolution features to avoid gradual disappearance of tiny object features. The study was performed on a public dataset of PCB defects with 693 images and was able to enhance detection of tiny defects with 98.90% mAP.

YOLO models have also shown useful for quality assurance tasks such as detection of small objects and components in images of products and scenes. A modified version of YOLOv4 incorporating feature fusion of shallow layers achieved mAP of 86% in identifying defects of electronic chips surface over 896 inspection images. A study in camera calibration on 141 checkerboard images (augmented to total of 2810 images), made use of an improved YOLOX model to perform component and checker-board corner detection. The study achieved better accuracy and robustness than traditional methods via modifying YOLOX by incorporating squeeze-and-excitation (SE) attention mechanism after the Spatial Pyramid Pooling (SPP) module to allow for improved localization and recognition of regions of interest while capturing position information. Another study in building a deep learning-based fabric inspection proposed an improved Yolov4 algorithm via enhanced SPP that used soft pooling instead of max pooling layer. Unlike max pooling that can miss the details, soft pooling selected the feature map elements in proportion to the probability of the values rather than the absolute values of elements. The study also adopted the contrast-limited adaptive histogram equalization (CLAHE) technique to improve image quality. Comparisons of the detection results between the YOLO models (both the original and the improved version) showed superior performance over Faster-RCNN approach with 86.5% mAP on VOC dataset.

The majority of object detection studies are based on models trained on COCO dataset or in-house datasets with up to 80 distinct classes of objects. Therefore, these object detection studies are either application based or are designed to predict a limited number of objects. One exception is YOLO9000 study which can detect over 9000 objects but with a performance that suffers from introducing a large number of classes in a single model (mAP of 19.7 on ImageNet data, 16 on COCO dataset and 78.6 on VOC data).

In computer vision use cases like that described here, an object localization step precedes a layout verification where matching techniques compare features extracted from object images and an original refence image. The transformer-based approach of LoFTR has shown improved performance compared to the conventional approaches such as Scale Invariant Feature Transform (SIFT). Unlike convolutional only approaches, the transformer used in LoFTR allows for reception and attention to both local neighborhood and global context for production of feature matches. LoFTR has been used in various fields for feature extraction and matching to enable localization. As an example, in building automatic electric power inspection tool using visual and thermal images, LoFTR was employed to extract and match feature points from RGB-Thermal images. A homography matrix calculated by random sample consensus (RANSAC) algorithm was then used to register RGB-Thermal images. A study in multi-object tracking in densely occupied scenes used LOFTR to enable extraction of both local and global image information for feature matching and localization purposes. The study interleaved self and cross attention blocks to leverage spatial relationships and temporal relationship from self and cross attention and enable modeling of multi-object tracking and interaction. Another study in inspection of electrical products, used LoFTR to address the registration problem of images of electrical equipment captured from two different image domains (visible and infrared). Progressive sample consensus (PROSAC) was then used to estimate a transformation matrix between the matched features. The approach showed satisfactory performance results with robustness to variations, weak-texture regions, or repetitive patterns by taking advantage of long-range context. A study in automated bridge defect detection utilized LoFTR to establish relationship and matched features between bridge images and their repair maps resulting in significant improvement (13× increase) of accuracy over traditional approaches such as SIFT.

Scientists have used YOLO models along with LoFTR technique for object detection and image layout verification in various use cases. For example, one study proposed a framework to localize vehicles in a parking lot by firstly finding the closest match for the location of a query image through finding correspondent points between a query image and a parking lot image database by LoFTR. Then, YOLOv5 detected vehicles and removed similar vehicle match points to identify the location with improved precision. The comparison with traditional techniques such as SIFT showed that LoFTR coupled with YOLOv5 trained by an in-house database of vehicle images in parking lot demonstrated capabilities of finding correspondence at an accuracy of 86.9%.

Similarly, a perception system coupled YOLOX model with LoFTR to perceive and layout environment under suboptimal image quality scenarios. Firstly, YOLOX localized objects in the enhanced images; secondly, LoFTR obtained the corresponding pixel points to identify distant objects as outliers in the matched feature map. Their experiments showed robust and accurate performance in understanding the environment, detecting pedestrians and vehicles in poor-quality scenarios (such as low light and noise), and small object images.

### SUMMARY

According to one aspect, an instrument tray inspection includes a user interface adapter, a recognition engine, recognition post-processor, and a tray layout verifier. The user interface adapter is configured to receive a test image of an instrument tray and determine a tray identifier. The tray identifier is visually indicated on the instrument tray. The recognition engine is configured to generate a plurality of object predictions from the test image with a trained object recognition model. Each of the plurality of object predictions comprises a predicted location of a component within the instrument tray and a component identifier. The recognition post-processor is configured to post-process the plurality of object predictions with non-max suppression based on a predetermined tray configuration associated with the tray identifier. The predetermined tray configuration comprises a plurality of expected component identifiers. The tray layout verifier is configured to determine whether the plurality of object predictions match a predetermined tray layout associated with the tray identifier, and to clear the instrument tray for re-use in response to a determination that the plurality of object predictions match the predetermined tray layout associated with the tray identifier. In an embodiment, the tray layout verifier is further configured to flag the instrument tray for further inspection in response to a determination that the plurality of object predictions do not match the predetermined tray layout associated with the tray identifier.

In an embodiment, the user interface adapter configured to receive the test image comprises a user interface adapter configured to receive the test image from a user device. In an embodiment, the user interface adapter is further configured to transmit a user interface indicative of whether the plurality of object predictions match the predetermined tray layout associated with the tray identifier to the user device. In an embodiment, the user interface adapter configured to determine the tray identifier comprises a user interface adapter configured to receive the tray identifier from the user device. In an embodiment, the user interface adapter configured to determine the tray identifier comprises a user interface adapter configured to recognize the tray identifier in the test image.

In an embodiment, the recognition post-processor configured to post-process the plurality of object predictions comprises a recognition post-processor configured to filter object predictions based on the plurality of expected component identifiers of the predetermined tray configuration. In an embodiment, the recognition post-processor configured to post-process the plurality of object predictions further comprises a recognition post-processor configured to perform non-max suppression based on the plurality of expected component identifiers in response to filtering of the object predictions. In an embodiment, the predetermined tray configuration further comprises an expected quantity for each expected component identifier. The recognition post-processor configured to post-process the plurality of object predictions further comprises a recognition post-processor configured to remove any object predictions having an associated expected quantity value that is less than one. In an embodiment, the recognition post-processor configured to post-process the plurality of object predictions further comprises a recognition post-processor configured to select the expected quantity of non-overlapping, highest-confidence object predictions for each expected component identifier having an associated expected quantity greater than one.

In an embodiment, the predetermined tray layout comprises a plurality of reference object identifications. Each reference object identification comprises an expected location of a component within the instrument tray and an expected component identifier. The tray layout verifier configured to determine whether the plurality of object predictions match the predetermined tray layout associated with the tray identifier comprises a tray layout verifier configured to: register the test image and a reference image associated with the tray identifier to generate a transformation matrix; to transform the expected locations of the reference object identifications of the predetermined tray layout with the transformation matrix; to compare each reference object identification of the transformed predetermined tray layout to a corresponding object prediction of the plurality of object predictions; and to determine a presence indicator and a correct placement indicator for each reference object identification in response to a comparison of each reference object identification to the corresponding object prediction. In an embodiment, the tray layout verifier configured to compare each reference object identification to the corresponding object prediction comprises a tray layout verifier configured to determine whether an expected location of the reference object identification matches a predicted location of the corresponding object prediction within a predetermined threshold and to determine whether an expected component identifier of the reference object identification matches a component identifier of the corresponding object prediction. In an embodiment, the tray layout verifier configured to determine whether the expected location of the reference object identification matches the predicted location of the corresponding object prediction within the predetermined threshold comprises a tray layout verifier configured to determine whether a first centroid of a first bounding box of the expected location is within a predetermined percentage of a second centroid of a second bounding box of the predicted location.

According to another aspect, a method for instrument tray inspection comprises receiving, by a computing device, a test image of an instrument tray; determining, by the computing device, a tray identifier, wherein the tray identifier is visually indicated on the instrument tray; generating, by the computing device, a plurality of object predictions from the test image with a trained object recognition model, wherein each of the plurality of object predictions comprises a predicted location of a component within the instrument tray and a component identifier; post-processing, by the computing device, the plurality of object predictions with non-max suppression based on a predetermined tray configuration associated with the tray identifier, wherein the predetermined tray configuration comprises a plurality of expected component identifiers; determining, by the computing device, whether the plurality of object predictions match a predetermined tray layout associated with the tray identifier; and clearing, by the computing device, the instrument tray for re-use in response to determining that the plurality of object predictions match the predetermined tray layout associated with the tray identifier.

In an embodiment, receiving the test image comprises receiving the test image from a user device, and the method further comprises generating, by the user device, a user interface indicative of whether the plurality of object predictions match the predetermined tray layout associated with the tray identifier.

In an embodiment, post-processing the plurality of object predictions comprises filtering object predictions based on the plurality of expected component identifiers of the predetermined tray configuration.

In an embodiment, the predetermined tray layout comprises a plurality of reference object identifications. Each reference object identification comprises an expected location of a component within the instrument tray and an expected component identifier. Determining whether the plurality of object predictions match the predetermined tray layout associated with the tray identifier comprises registering the test image and a reference image associated with the tray identifier to generate a transformation matrix; transforming the expected locations of the reference object identifications of the predetermined tray layout with the transformation matrix; comparing each reference object identification of the transformed predetermined tray layout to a corresponding object prediction of the plurality of object predictions; and determining a presence indicator and a correct placement indicator for each reference object identification in response to comparing each reference object identification to the corresponding object prediction.

According to another aspect, one or more non-transitory, computer readable storage media comprise a plurality of instructions that, in response to being executed, cause a computing device to receive a test image of an instrument tray; determine a tray identifier, wherein the tray identifier is visually indicated on the instrument tray; generate a plurality of object predictions from the test image with a trained object recognition model, wherein each of the plurality of object predictions comprises a predicted location of a component within the instrument tray and a component identifier; post-process the plurality of object predictions with non-max suppression based on a predetermined tray configuration associated with the tray identifier, wherein the predetermined tray configuration comprises a plurality of expected component identifiers; determine whether the plurality of object predictions match a predetermined tray layout associated with the tray identifier; and clear the instrument tray for re-use in response to determining that the plurality of object predictions match the predetermined tray layout associated with the tray identifier.

In an embodiment, to post-process the plurality of object predictions comprises to filter object predictions based on the plurality of expected component identifiers of the predetermined tray configuration. In an embodiment, to post-process the plurality of object predictions further comprises to perform non-max suppression based on component identifier in response to filtering the object predictions; and select an expected quantity of non-overlapping, highest-confidence object predictions for each expected component identifier having an associated expected quantity greater than one, wherein the predetermined tray configuration further comprises the expected quantity for each expected component identifier.

In an embodiment, the predetermined tray layout comprises a plurality of reference object identifications. Each reference object identification comprises an expected location of a component within the instrument tray and an expected component identifier. To determine whether the plurality of object predictions match the predetermined tray layout associated with the tray identifier comprises to register the test image and a reference image associated with the tray identifier to generate a transformation matrix; to transform the expected locations of the reference object identifications of the predetermined tray layout with the transformation matrix; to compare each reference object identification of the transformed predetermined tray layout to a corresponding object prediction of the plurality of object predictions; and to determine a presence indicator and a correct placement indicator for each reference object identification in response to comparing each reference object identification to the corresponding object prediction.

### BRIEF DESCRIPTION OF THE DRAWINGS

The detailed description particularly refers to the following figures, in which:

FIG. 1 is a simplified block diagram of at least one embodiment of a system for automated orthopaedic surgical instrument tray inspection;

FIG. 2 is a simplified block diagram of an environment that may be established by an inspection device of the system of FIG. 1;

FIG. 3 is a simplified flow diagram of at least one embodiment of a method for training a custom object recognition model that may be executed in connection with the system of FIG. 1;

FIGS. 4 and 5 are a simplified flow diagram of at least one embodiment of a method for automated surgical instrument tray inspection that may be executed by an inspection device of FIGS. 1-2; and

FIGS. 6-11 illustrate experimental results that may be achieved by the system of FIGS. 1-2.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

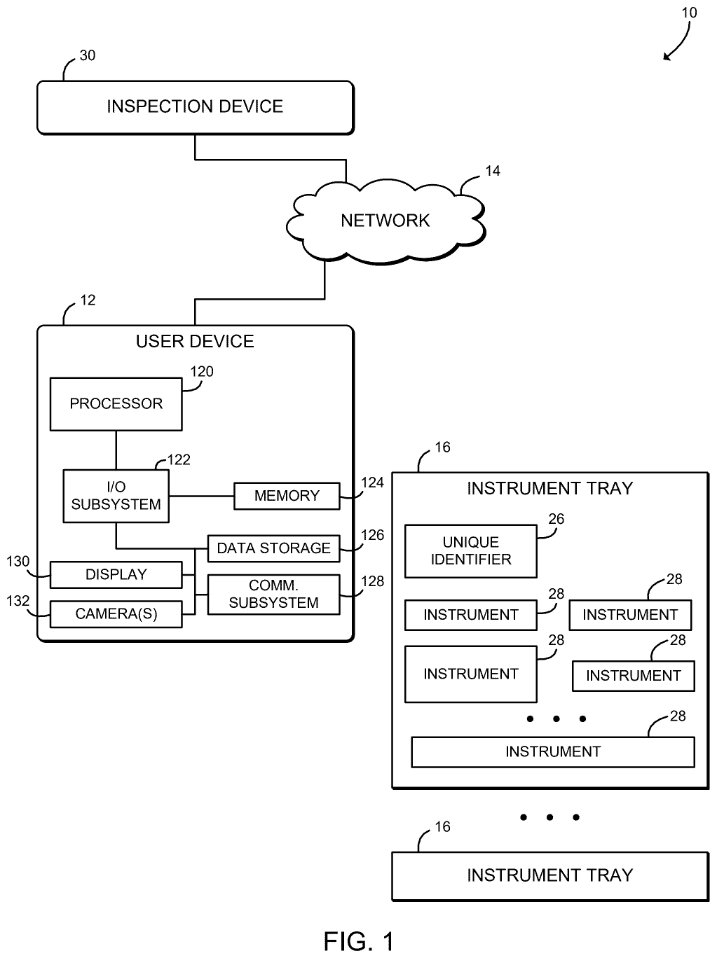

Referring now to FIG. 1, an end-to-end inspection system 10 for orthopedic surgical instrument trays is disclosed. In this example, the trays are specifically focused on joint reconstruction procedures. The system 10 includes a user device 12 that is configured to communicate with an inspection device 30 over a network 14 to process scans of one or more instrument trays 16 and receive inspection results. In use, an operator of the user device 12 may scan a unique identifier 26 (illustratively, a barcode) on each tray 16, capture an image of the tray 16 and its contents, transmit the image to the inspection device 30, and receive the inspection results within approximately one second. As described in greater detail below, the system 10 includes a cloud-based inspection module or device 30 that processes test images and generates the inspection results.

Quality control and product integrity are important functions for supply chain and industrial processes. Typical manual inspections are costly, labor-intensive, and time-consuming. The disclosed system 10 provides an automated visual inspection tool, powered by novel computer vision technologies described herein, and ensures consistency in inspection, and further offers fast and reliable inspection and quality assessment. Compared to existing automated systems, the disclosed system 10 provides improved performance (e.g., improved compute efficiency and/or improved recognition accuracy) with a large number of classes of objects. As described further below, the disclosed system 10 enables detection of over 1000 classes of objects (e.g., surgical instruments or other tray components) using a single object recognition model, allowing straightforward maintenance and reducing maintenance and infrastructure cost for industrial applications. The disclosed system 10 provides scalability, as it has the capability to expand classification to accommodate new surgical trays and components as new datasets become available for retraining. The disclosed system 10 offers a light-weight pipeline that can be deployed within a wide range of industrial settings and, for example, be used on mobile and tablet devices to generate responses in under 1 second, making it practical to be used on the go or in an industrial production setting while handling surgical trays. Additionally, in testing, positive feedback was received from users, which indicated that utilizing the disclosed system 10 has helped reduce the end-to-end inspection time by at least 40% as compared to previous processes.

Referring again to FIG. 1, the user device 12 may be embodied as any type of device capable of performing the functions described herein. For example, a user device 12 may be embodied as, without limitation, a tablet computer, a smartphone, a laptop computer, a desktop computer, a workstation, a network appliance, a web appliance, a consumer electronic device, a distributed computing system, a multiprocessor system, and/or any other computing device capable of performing the functions described herein. As shown in FIG. 1, the illustrative user device 12 includes a processor 120, an I/O subsystem 122, memory 124, a data storage device 126, and a communication subsystem 128. Of course, the user device 12 may include other or additional components, such as those commonly found in a tablet computer (e.g., various input/output devices), in other embodiments. Additionally, in some embodiments, one or more of the illustrative components may be incorporated in, or otherwise form a portion of, another component. For example, the memory 124 and/or data storage 126, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the user device 12 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the user device 12. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the user device 12, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the user device 12 may be embodied as any communication circuit, device, or collection thereof, capable of enabling communications between the user device 12, the inspection device 30, and other remote devices. The communication subsystem 128 may be configured to use any one or more communication technology (e.g., wireless or wired communications) and associated protocols (e.g., Ethernet, Bluetooth®, Wi-Fi®, WiMAX, 3G LTE, 5G, etc.) to effect such communication.

As shown in FIG. 1, the user device 12 further includes a display 130 and one or more cameras 132. The display 130 may be embodied as any type of display capable of displaying digital images or other information, such as a liquid crystal display (LCD), a light emitting diode (LED), a plasma display, a cathode ray tube (CRT), or other type of display device. As described further below, the display 130 of the user device 12 presents a user interface indicative of tray layout verification results. In an embodiment, the user interface graphically indicates whether each component 28 of the instrument tray 16 is correctly placed (e.g., with a green outline) or is missing or misplaced (e.g., with a red outline around the expected location). The graphical user interface may be generated by the user device 12, by the inspection device 30, or by a combination of the user device 12 and the inspection device 30. For example, in an embodiment, the inspection device 30 may generate graphical elements and web elements (e.g., interactive markup elements), or other user interface elements and transmit those user interface elements to the user device 12 for rendering. As another example, in some embodiments the inspection device 30 may transmit data indicative of tray layout verification results to the user device 12, and the user device 12 may generate the user interface, for example using a native application, a web application, or other locally executed application. In some embodiments, the display 130 may be coupled to a touch screen to allow user interaction with the user device 12.

Each of the one or more cameras 132 may be embodied as a digital camera or other digital imaging device integrated with the user device 12 or otherwise communicatively coupled thereto. The camera 132 includes an electronic image sensor, such as an active-pixel sensor (APS), e.g., a complementary metal-oxide-semiconductor (CMOS) sensor, or a charge-coupled device (CCD). The camera 132 may be used to capture image data including, in some embodiments, capturing still images or video images.

The inspection device 30 be embodied as any type of computation or computer device capable of performing the functions described herein, including, without limitation, a server, a rack-mounted server, a blade server, a computer, a workstation, a laptop computer, a notebook computer, a tablet computer, a mobile computing device, a wearable computing device, a multiprocessor system, a network appliance, a web appliance, a distributed computing system, a processor-based system, and/or a consumer electronic device. Thus, the inspection device 30 includes components and devices commonly found in a computer or similar computing device, such as a processor, an I/O subsystem, a memory, a data storage device, and/or communication circuitry. Those individual components of the inspection device 30 may be similar to the corresponding components of the user device 12, the description of which is applicable to the corresponding components of the inspection device 30 and is not repeated herein so as not to obscure the present disclosure. Additionally, in some embodiments, the inspection device 30 may be embodied as a “virtual server” formed from multiple computing devices distributed across the network 14 and operating in a public or private cloud. Accordingly, although the inspection device 30 is illustrated in FIG. 1 as embodied as a single computing device, it should be appreciated that the inspection device 30 may be embodied as multiple devices cooperating together to facilitate the functionality described below.

As discussed further below, the user device 12 and the inspection device 30 may be configured to transmit and receive data with each other and/or other devices of the system 10 over the network 14. The network 14 may be embodied as any number of various wired and/or wireless networks. For example, the network 14 may be embodied as, or otherwise include, a wired or wireless local area network (LAN), a wired or wireless wide area network (WAN), a cellular network, and/or a publicly-accessible, global network such as the Internet. As such, the network 14 may include any number of additional devices, such as additional computers, routers, stations, and switches, to facilitate communications among the devices of the system 10.

As shown, the user device 12 may be used to scan and capture images of one or more surgical instrument trays 16. Each of the trays 16 includes a visible or otherwise accessible unique identifier 26, which may be scanned by the user device 12. For example, the unique identifier 26 may be embodied as a bar code or other visible indicator of the unique identifier, a radio frequency identifier (RFID) tag, or other identifying tag.

Each tray 16 is configured to store one or more surgical instruments 28. The instruments 28 may include, for example, one or more, or any combination of, surgical reamers, broaches, impactors and impaction handles, prosthetic trial components, trial liners, drill guides, cutting blocks, surgical saws, ligament balancers, or other medical devices used in the performance of an orthopaedic surgical procedure (e.g., joint replacement surgery or another orthopaedic procedure). Additionally or alternatively, the tray 16 may be embodied as a trauma surgical trays or another type of surgical tray. Each tray 16 may include multiple compartments, molded features, component holders, or other features configured to retain or otherwise store a particular instrument 28 in a particular location and with a particular orientation. Accordingly, each type of tray 16 may be associated with a specific configuration of type, quantity, and location of instruments 28. As described further below, the system 10 is trained to recognize and validate tray layouts for many types of trays 16 (e.g., dozens of types of trays). Additionally, each tray 16 may include a complicated arrangement of potentially overlapping instruments 28, and many of the instruments 28 are identical or very similar. For example, a tray 16 may include multiple similar instruments 28 having different sizes (e.g., trial components, broaches, etc.), multiple instances of the same instrument 28, and other combinations. As described further below, in some embodiments, the system 10 may be trained to recognize a large number of distinct instruments (e.g., over 1000 types of instruments).

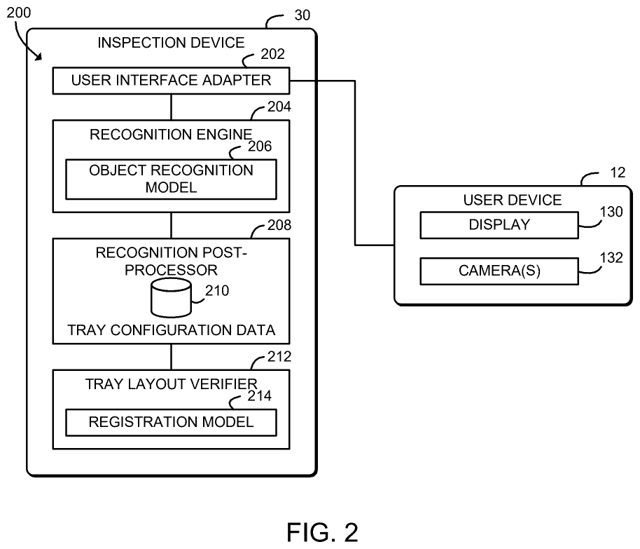

Referring now to FIG. 2, in the illustrative embodiment, the inspection device 30 establishes an environment 200 during operation. The illustrative environment 200 includes a user interface adapter 202, a recognition engine 204, a recognition post-processor 208, and a tray layout verifier 212, which may be embodied as hardware, firmware, software, or a combination thereof. As such, in some embodiments, one or more of the components of the environment 200 may be embodied as circuitry or a collection of electrical devices (e.g., user interface adapter circuitry 202, recognition engine circuitry 204, recognition post-processor circuitry 208, and/or tray layout verifier circuitry 212). It should be appreciated that, in such embodiments, one or more of those components may form a portion of the processor, the memory, the data storage, the I/O subsystem, the communication subsystem, and/or other components of the inspection device 30. Additionally or alternatively, it should be understood that, as described above, one or more components of the environment 200 may be formed with multiple computing devices distributed across the network 14 and operating in a public or private cloud.

The user interface adapter 202 is configured to receive a test image of an instrument tray 16 and to determine a tray identifier 26. The test image may be received from a user device 12. The tray identifier 26 is illustratively visually indicated on the instrument tray 16. The tray identifier 26 may be received from the user device 12 or may be recognized in the test image.

The recognition engine 204 is configured to generate multiple object predictions from the test image using a trained object recognition model 206. Each of the object predictions includes a predicted location of a component within the instrument tray and a component identifier.

The object recognition model 206 may be embodied as a trained machine learning model for object recognition and classification. In particular, the object recognition model 206 may be configured or otherwise tuned for real-time or near-real-time performance. For example, the object recognition model 206 may be embodied as a single-pass convolutional neural network such as a You Only Look Once (YOLO) model. Illustratively, the model 206 may be embodied as a YOLOv7-X model, with a default loss function or a custom loss function as described further below. Of course, other YOLO models (e.g., YOLOv8, YOLOv6, or another model) may be used in other embodiments. Additionally or alternatively, in some embodiments the object recognition model 206 may be embodied as a two-pass model such as Faster-RCNN or another R-CNN model.

The recognition post-processor 208 is configured to post-process the object predictions with non-max suppression (NMS) based on predetermined tray configuration data 210 associated with the tray identifier 26. The predetermined tray configuration data 210 includes multiple expected component identifiers associated with each tray identifier 26, and may include an expected quantity for each expected component identifier. Post-processing the object predictions may include filtering object predictions based on the expected component identifiers of the tray configuration data 210. Such filtering may include performing non-max suppression based on component identifier to identify object predictions that are in excess of an expected quantity value. The filtering may further include selecting the expected quantity of non-overlapping, highest-confidence object predictions for each expected component identifier having an associated expected quantity value greater than one.

The tray layout verifier 212 is configured to determine whether the object predictions match a predetermined tray layout associated with the tray identifier 26. The predetermined tray layout includes multiple reference object identifications. Similar to the object predictions, each reference object identification includes an expected location of a component within the instrument tray and an expected component identifier. The tray layout verifier is further configured to clear the instrument tray 16 for re-use in response to determining that the object predictions match the predetermined tray layout, and may be further configured to flag the instrument tray 16 for further inspection in response to determining that the object predictions do not match the predetermined tray layout.

Determining whether the object predictions match the predetermined tray layout associated with the tray identifier includes registering the test image and a reference image associated with the tray identifier 26 to generate a transformation matrix. Registration may be performed with a registration model 214, which may be embodied as a pre-trained machine learning model for matching keypoints between images, such as LoFTR. Determining whether the object predictions match the predetermined tray layout further includes transforming the expected locations of the reference object identifications of the predetermined tray layout with the transformation matrix; comparing each reference object identification of the transformed predetermined tray layout to a corresponding object prediction of the plurality of object predictions; and determining a presence indicator and a correct placement indicator for each reference object identification. Comparing each reference object identification to the corresponding object prediction may include determining whether an expected location of the reference object identification matches a predicted location of the corresponding object prediction within a predetermined threshold and determining whether an expected component identifier of the reference object identification matches a component identifier of the corresponding object prediction. Determining whether the expected location of the reference object identification matches the predicted location of the corresponding object prediction within the predetermined threshold may include determining whether a centroid of a bounding box of the expected location is within a predetermined percentage of a centroid of a bounding box of the predicted location.

The user interface adapter 202 may be further configured to generate a user interface or transmit a user interface to the user device 12. The user interface is indicative of whether the plurality of object predictions match the predetermined tray layout associated with the tray identifier to the user device 12.

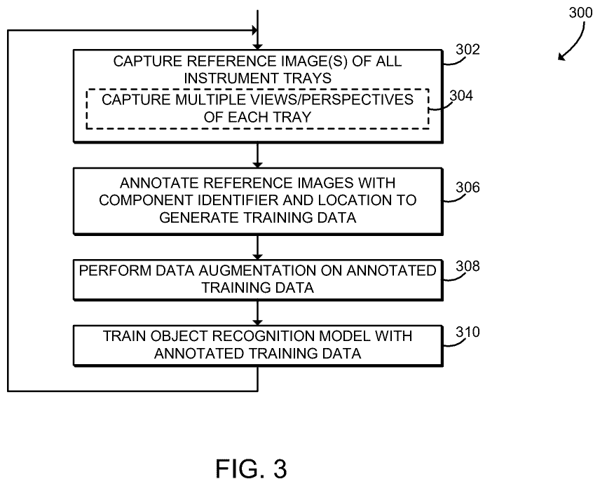

Referring now to FIG. 3, in use, a computing device such as the inspection device 30 may execute a method 300 for training a custom object recognition model. The method 300 begins with block 302, in which the computing device captures reference image(s) of all types of surgical instrument trays 16 that will be inspected, including all instruments 28 and other components located in their correct positions. In some embodiments, in block 304 the computing device may capture multiple views or perspectives of each tray 16. For example, the computing device may extract multiple video frames with different perspectives of the tray 16 from a captured video.

In block 306, the reference images are annotated with component identifier and location for each contained instrument 28 to generate training data. Each component identifier may be embodied as a name, a number, a code, or another unique identifier assigned to a particular type of component (e.g., instrument 28 or other component) stored in the tray 16. The location may be embodied as a bounding box or other indication of storage location for that component within the tray 16. The bounding box may be defined relative to the reference image for the tray 16. As described further below, a registration process will be used to align the reference image to a test image.

The annotation data generated for a reference image may be used to generate tray configuration data 210 for the associated instrument tray 16. For example, the annotation data may be converted to a configuration file format that may be processed by the inspection device 30 as described further below.

In block 308, the computing device may perform data augmentation on the training data to increase the amount of training data. For example, additional training images may be generated from the captured reference images using operations such as flip, rotation, hue, saturation, and value (HSV) change, translate, scale, and mosaic.

In block 310, the computing device trains the object recognition model 206 with the annotated and augmented training data. The computing device may use any appropriate training algorithm. For example, the computing device may randomly partition the training data into training and validation subsets. The model 206 may be initialized with publicly available pre-trained weights, and then training may be performed with the training subset until a desired overall mean average precision (mAP) is achieved, as measured with the verification subset.

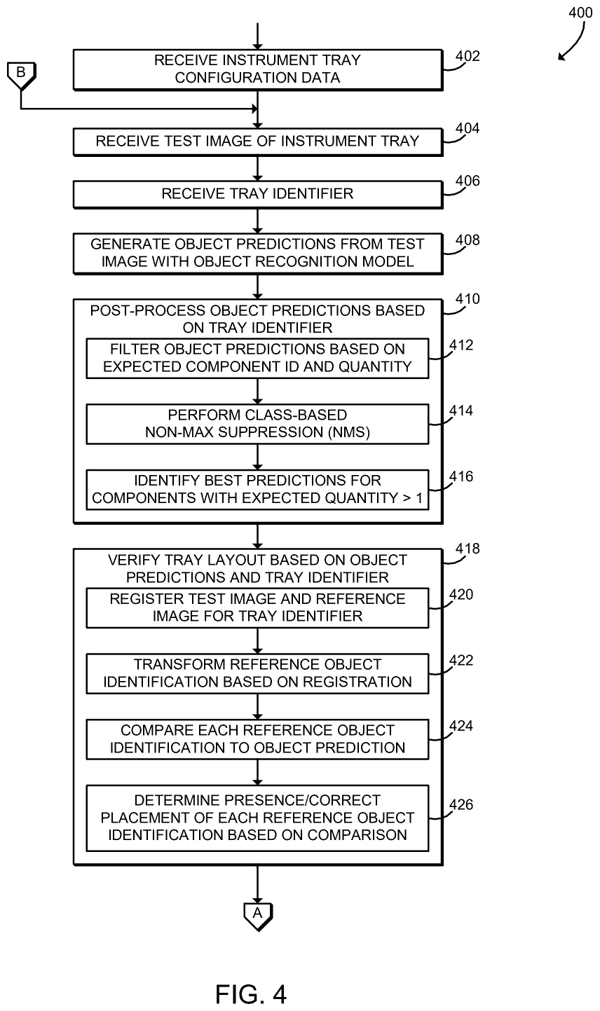

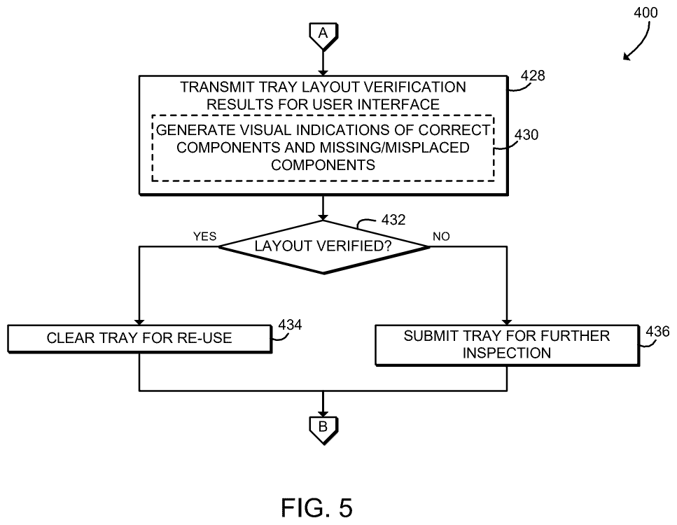

After training is complete, the trained object recognition model 206 may be provided to the inspection device 30. The trained object recognition model 206 may be used to perform tray inspections as described below in connection with FIGS. 4 and 5. The method 300 may loop back to block 302 to gather additional training data and perform additional training. For example, training may be performed periodically, after certain new trays 16 have been introduced, after certain new instruments 28 have been introduced, or at other times. As discussed above, the method 300 may be performed by the inspection device 30 or another device. For example, the method 300 may be performed using a cloud instance with GPU support or other available AI hardware.

Referring now to FIGS. 4-5, in use, the inspection device 30 may execute a method 400 for automated surgical instrument tray inspection. It should be appreciated that, in some embodiments, the operations of the method 400 may be performed by one or more components of the environment 200 of the inspection device 30 as shown in FIG. 2. The method 400 begins with block 402, in which the inspection device 30 receives instrument tray configuration data 210. The instrument tray configuration data 210 indicates the component identifier and quantity for each instrument 28 or other component included in a tray 16. The instrument tray configuration data 210 may be indexed or otherwise searchable by tray identifier. As described above, in the illustrative embodiment the instrument tray configuration data 210 is generated based on annotations of training data used to train the object recognition model 206.

In block 404, the inspection device 30 receives one or more test images from the user device 12. The test images include digital images, video frames, or other images of an instrument tray 16 that is being inspected. The test image is captured using a camera 132 of the user device 12. In other configurations of the device, the test image may be captured using additional cameras or another image capture devices.

In block 406, the inspection device 30 receives a tray identifier 26 for the instrument tray 16 being inspected. The tray identifier 26 is received from the user device 12. For example, the user device 12 scans a bar code or other visual indicator attached to the tray 16 using a camera or dedicated bar code reader and then transmits the indicated tray identifier 26 to the inspection device 30. As another example, the user device 12 may scan an RFID tag or other electronic identifier of the tray 16 and then transmit the corresponding tray identifier to the inspection device 30. Additionally or alternatively, in some embodiments the inspection device 30 may determine the tray identifier 26. For example, the inspection device 30 may recognize a bar code or other identifier in the test image received from the user device 12.

In block 408, the inspection device 30 generates object predictions from the test image using the object recognition model 206. To do so, the inspection device 30 submits the test image as an input to the model 206 and receives a set of object predictions as an output. Each object prediction indicates a predicted location for a recognized instrument 28 or other component along with a corresponding predicted component identifier. The predicted location is embodied as a bounding box relative to the test image and is associated with a confidence value generated by the model 206. It should be appreciated that other coordinates may embody the predicted location in other configurations of the device 30. The confidence value may also be omitted in other configurations.

In block 408, the inspection device 30 post-processes the object predictions based on the received tray identifier 26. Post-processing the object predictions includes removing duplicate object predictions, removing incorrect object predictions, and selecting higher-confidence object predictions. In other embodiments, post-processing may include one or more additional operations for otherwise improving accuracy of the object predictions.

Illustratively, post-processing starts in block 412, in which the inspection device 30 filters the object predictions based on expected component identifier and quantity for the received tray identifier 26. Filtering the object predictions removes any object predictions that do not match an expected component identifier associated with the tray identifier 26. For example, the inspection device 30 may look up a list of expected component identifiers for the received tray identifier 26 from the tray configuration data 210. The inspection device 30 may remove any object prediction including a component identifier that is not included in the list of expected component identifiers. Similarly, in some embodiments, the inspection device 30 may determine an expected quantity for each component identifier, and remove object predictions associated with component identifiers that exceed the expected quantity.

In block 414, the inspection device 30 performs class-based non-max suppression (NMS). Illustratively, for each expected component identifier (i.e., class), the inspection device 30 identifies all non-overlapping object predictions for that component identifier (e.g., using an intersection over union metric), and retains the object prediction having the highest associated confidence level.

In block 416, the inspection device 30 identifies the best predictions for component identifiers with an expected quantity greater than one. For example, the inspection device 30 identifies non-overlapping object predictions for a particular component identifier and selects the expected quantity of those non-overlapping object predictions having the highest associated confidence score. Non-overlapping object predictions may be identified, for example, as those with bounding boxes having an intersection over union (IoU) value less than a predetermined threshold, such as 0.5.

After post-processing, in block 418 the inspection device 30 verifies the tray layout based on the object predictions and the received tray identifier. Verifying the tray layout includes identifying any missing or misplaced components in the tray 16 currently being inspected. That is, verifying the tray layout includes determining whether every expected component associated with the received tray identifier 26 has been recognized in the test image, and determining whether every recognized component is in the expected location and orientation. The expected component identifier and the expected location for each component in the reference tray may be stored or otherwise referenced in a collection of reference object identifiers, which may be generated during training as described above.

Verification of the tray layout begins in block 420, in which the inspection device 30 registers the test image and a reference image for the received tray identifier 26. The reference image is illustratively one of the reference images captured during training, as described above in connection with FIG. 3. Registering the test image and the reference image includes aligning matching keypoints from the reference image to the test image. Registration is performed using the registration model 214, which is illustratively a pre-trained machine learning model such as a LOFTR model. Using predicted matching keypoints from the registration model 214, the inspection device 30 generates a transformation matrix (e.g., a homography matrix) that can be used to align the reference image to the test image. The transformation matrix is generated by solving a regression problem, for example using a variation of the RANSAC algorithm for model fitting, such as USAC-MAGSAC.

In block 422, the inspection device 30 transforms reference object identifications based on the registration determined in block 420. As described above, the reference object identifications include expected component identifiers and expected locations (e.g., bounding boxes) for each component identifier. The expected locations are relative to the reference image. For example, the reference object identifications may be embodied as or based on training annotation data, ground truth data, or other classification data associated with the reference image. The reference object identifications are transformed by applying the transformation matrix determined in block 420 to the bounding box associated with each reference object identification.

In block 424, the inspection device 30 compares each transformed reference object identification to the object predictions. Illustratively, the inspection device 30 compares the transformed location of each reference object identification to the location of one or more object predictions. Those locations are compared in the illustrative embodiment by determining a distance between centroids of corresponding bounding boxes. Distance may be determined relative to the size of the bounding box or using any other appropriate metric. As another example, to compare locations, bounding boxes may be checked for overlap, for example, using IoU or another technique. Further, the expected component identifier of the reference object identification is compared to the predicted component identifier of one or more object predictions.

In block 426, the inspection device 30 determines the presence (e.g., present or missing) and the placement (e.g., correctly placed or incorrectly placed) for each reference object identification based on the comparison of block 424. The inspection device 30 identifies any missing components, which include expected component identifiers in the reference object identification that are not included in any of the object predictions. Additionally, the inspection device 30 identifies any misplaced components, which include object predictions with a component identifier that matches an expected component identifier but with a location that does not match an expected location for that component identifier. Further additionally, the inspection device 30 identifies correctly placed components, which are those object predictions with both a component identifier and a location that matches the expected component identifier and the expected location for a reference object identification.

In block 428, shown in FIG. 5, the inspection device 30 transmits tray layout verification results determined in block 418 to the user device 12 for presentation in a user interface. The user interface indicates whether the tray layout was successfully verified, and may identify any missing or misplaced components in the tray 16, as well as any correctly placed components. As described above, the graphical user interface may be generated by the user device 12, by the inspection device 30, or by a combination of the user device 12 and the inspection device 30. In some embodiments, in block 430 the inspection device 30 may generate one or more visual indications of correctly placed components and missing or misplaced components. For example, the inspection device 30 may generate an image, a graphical element, an interactive application, a web application, or other user interface and transmit that user interface to the user device 12. Additionally or alternatively, in some embodiments the user device 12 itself may generate part or all of the user interface based on tray layout verification results data provided by the inspection device 30. For example, a native application executed by the user device 12 may render a visual indication of the tray layout verification results received from the inspection device 30.

In the illustrative embodiment, the visual interface presents the captured test image with visually overlayed, color-coded annotations identifying correctly placed components as well as missing or misplaced components. Each annotation is illustratively an outline corresponding to the bounding box of an expected location of a component, and is color-coded based on whether the component is correctly placed or not. For example, correctly placed components may be outlined in green, and the expected location of missing or misplaced components may be outlined in red. Additionally, each annotation may provide a component identifier or other indication of the expected component at that location. Of course, in other embodiments the annotations may be visually distinguished using other techniques such as patterns, shadings, icons, or other techniques.

In block 432, the inspection device 30 determines whether the tray layout was successfully verified. Illustratively, the inspection device 30 determines whether every expected component identifier and expected location of the reference object identifications successfully matches a corresponding object prediction, as described above. As an additional example, the inspection device 30 may determine whether any missing or misplaced components were detected. If the tray layout was successfully verified, the method 400 branches to block 434, in which the tray 16 being inspected is cleared for re-use. For example, the inspection device 30 may send a notification to the user device 12 or another device indicating that the particular tray 16 has been successfully verified. After clearing the tray for re-use, the method 400 loops back to block 404, shown in FIG. 4, to continue inspecting additional instrument trays 16. Referring again to block 432, if the tray layout was not successfully verified, the method 400 branches to block 436, in which the tray 16 being inspected is submitted for further inspection. For example, the inspection device 30 may send a notification to the user device 12 or another device indicating that the particular tray 16 has not been successfully verified, and may enter the tray 16 into a queue or otherwise indicate further inspection is required. After flagging the tray for additional inspection, the method 400 loops back to block 404, shown in FIG. 4, to continue inspecting additional instrument trays 16.

### EXAMPLES

As an illustration of one or more embodiments of the system 10 described above, a study was performed in which various illustrative embodiments of the system 10 were constructed, trained, and tested.

### Data Sources

### Training and Validation Data

The study dataset included 74 surgical trays utilized in joint reconstruction procedures, with a total of 1039 unique components (classes). Video recordings of each tray were captured from a consistent distance of approximately 30 cm above the tray to prepare a dataset for training an object detection model 206. The camera used a gentle oscillating motion within a range of +/−5 degrees to capture diverse perspectives of the trays. For each tray, comprising all components correctly positioned, two separate videos were recorded using different cameras, from two orientations (0 degree and 180 degrees). Each video was approximately 50 seconds long with a 30 frames per second (FPS) rate, resulted in an average of 3000 frames per tray. The video frames were subsequently extracted and annotated as a primary dataset for model training and validation.

### Test Data

An informal user acceptance testing (UAT) approach was employed to assess the performance of the trained object detection model. A random subset was selected comprising ten trays for evaluation from the total pool of 74 training trays, and realistic test scenarios were selected, representing a variety of practical situations in which end users may employ the end-to-end ML inspection solution. This testing methodology was implemented to comprehensively evaluate the model's ability to detect objects across a range of conditions, thereby providing insights into its robustness and suitability for industrial applications. Therefore, for the selected 10 trays, new static images were captured under 12 distinct scenarios, as outlined in Table 1. A total of 139 test images were collected to evaluate the performance of the end-to-end pipeline.

TABLE 1
Description of the 12 test scenarios that were used
to evaluate the developed end-to-end pipeline.

| Scenario | |
|---|---|
| number | Description |
| 1 | Similar looking components swapped |
| 2 | Components at wrong places |
| 3 | Components missing from the tray |
| 4 | Foreign components in the tray |
| 5 | Components that have changed appearance |
| 6 | Different lighting condition |
| 7 | Components floating in the tray |
| 8 | Components placed in improper way (e.g., upside down, face |
| | down, etc.) |
| 9 | Components at right places |
| 10 | Tray inspection at varied height |
| 11 | Images at varied angle |
| 12 | Tray placed at non-designated location |

### Data Pre-Processing

The data preprocessing included extracting video frames, annotating objects within these frames, and randomly partitioning the data into training and validation datasets. The annotated video frames were consolidated, resulting in a dataset comprising 1039 classes that were used to train a single model 206. The training and validation datasets were randomly divided, allocating 85% of the annotated images for training and the remaining 15% for validation. This partitioning resulted in 131,741 training images and 23,249 validation images, encompassing data from all 74 trays with a total of 1039 unique components.

### Object Detection Models

The single-stage YOLO and two-stage object detection Faster-RCNN models outlined in Table 2, below, were trained and evaluated.

One of the key design choices in Faster R-CNN is the selection of the feature extractor backbone, which plays a crucial role in determining the model's performance and commonly used backbones include ResNet, and MobileNet. The Faster-RCNN object detection model was trained using two distinct backbones: ResNet-101 and MobileNet-V3 with Feature Pyramid Network (FPN).

In the implementation of YOLOv7 and YOLOv8, a FCNN (Fully Connected Neural Network) architecture was adopted. YOLOv7-X (with the original loss function as well as a custom loss function), YOLOv8-X, and YOLOv8-X6 were trained and evaluated. Given the presence of numerous objects with similar appearances but varying sizes or aspect ratios, a custom loss function was implemented, which was evaluated with YOLOv7-X model. This custom loss function incorporated an additional term into the original loss, accounting for the predicted bounding box area and aspect ratio using a smooth L1 loss. Furthermore, YOLOv8-X and YOLOv8-X6 models were trained and evaluated.

Data augmentation techniques were implemented in all model trainings including flip, rotation (+/−10 degrees), hue, saturation, and value (HSV), translate, scale, and mosaic. The weights of all models were initialized with publicly available pre-trained weights trained on COCO datasets. The models were trained for 20 epochs, at which the achieved mAP@0.5 on validation data was >0.99 on an Azure virtual machine with one V100 Nvidia GPU with 16 GB memory.

A custom Non-Max Suppression (NMS) algorithm was developed to filter the object detection predictions instead of applying a standard NMS. A single model was trained on consolidated data from 74 trays with a total of 1039 classes, and there were high visual similarities between some components across trays. Those factors would potentially increase the model false positive detections. To address this, a configuration file was created, based on training annotations, providing the list of component IDs belonging to each tray. In the first step, the post-processing algorithm utilizes the tray configuration data and enforces a tray-specific expected component IDs constraint on model predictions to filter out the predicted classes that do not belong to the tray under inspection. Next, a class-based NMS was applied to detections such that for each expected class (i.e., component ID), only the prediction with the highest confidence score is maintained, and the rest are removed from the predictions. In case the expected quantity of a component ID (of the same class) in a tray is greater than one, an Intersection over Union (IoU) condition is applied on top of the class-based NMS to keep the expected quantity of predictions that meet two criteria: they are non-overlapping (e.g., IoU<50%) and have the highest confidence scores among all predictions for the same class.

TABLE 2
Comparison of object detection models that
were trained and evaluated in this study.

| Architecture | Backbone | #Layers | #Param. | GFLOPs | Image |
|---|---|---|---|---|---|
| YOLOv7-X | E-ELAN | 467 | 77.8M | 211 | 640 |
| YOLOv8-X | C2f | 225 | 50.4M | 231 | 640 |
| YOLOv8-X6 | C2f | 477 | 98.7M | 267 | 1280 |
| Faster-RCNN | ResNet101 | 347 | 65.5M | 449 | 640 |
| Faster-RCNN | MobileNet- | 120 | 24.2 | 217 | 640 |
| | [symbol] | | | | |

[symbol]

indicates data missing or illegible when filed

The performance of each trained model was evaluated using the test data by measuring the mean Average Precision (mAP@50), accuracy, false positive rate, and false negative rate. The details of the end-to-end performance metrics are summarized in Table 3, below.

TABLE 3
The metrics used to evaluate the end-to-end (object detection
& layout verification) automated inspection pipeline.

| Metric | Definition |
|---|---|
| Correct | Predicted component ID = ground truth component ID |
| | AND |
| | Predicted position = ground truth position |
| Incorrect/ | Predicted component ID ≠ ground truth component ID |
| misclassified | OR |
| predictions | Predicted position ≠ ground truth position |
| Accuracy | Correct/total number of components in ground truth |
| | annotation |
| False | (Predicted present & ground truth missing)/total number |
| positive rate | of components in ground truth annotation |
| | OR |
| | (predicted in correct position & ground truth misplaced)/ |
| | total number of components in ground truth annotation |
| False | (Predicted missing & ground truth present)/total number |
| negative rate | of components in ground truth annotation |
| | OR |
| | (predicted in misplaced & ground truth in correct position)/ |
| | total number of components in ground truth annotation |

### Layout Verification

The tray layout verification goal was to verify the correct placement of individual components in their allocated slots in a tray and flag missing or misplaced components. Misplaced components refer to the components that are present in the tray but are not in their designated slots. Given the prior knowledge of the correct layout of components in a tray from the training data (reference image of the tray), and the predictions from an object detection model, the task of layout verification involves comparing the predicted position of each component versus the expected ones. A challenge in doing this comparison mainly arises from the differences between the reference or train images (video frames) and test images (static inspection images), including camera angle, camera-tray distance, rotation, component similarity, and lighting variations.

To co-register the reference training image and each test image a set of matching keypoints from two images is required. A pre-trained LoFTR model was adopted to extract the optimal matching keypoints between train (the first frame of the training video) and test images. A novel variation of the RANSAC algorithm, USAC-MAGSAC, was employed to solve a regression problem based on the predicted matching keypoints and infer the transformation matrix (homography matrix) and co-register the train and test image. Given the transformation matrix, all ground truth bounding boxes from the reference train image are translated into the test image, providing the anticipated position of each component within the test image. Subsequently, the predicted bounding boxes were compared with their corresponding expected bounding boxes. A predicted bounding box is classified as “correct” and displayed with a “green” box if its centroid location is within 20% of the expected position. Conversely, if the predicted location deviates from the expected position or if there is no prediction for an expected component, that component is marked as “missing/misplaced” and depicted with a red bounding box in the expected location.

### Results

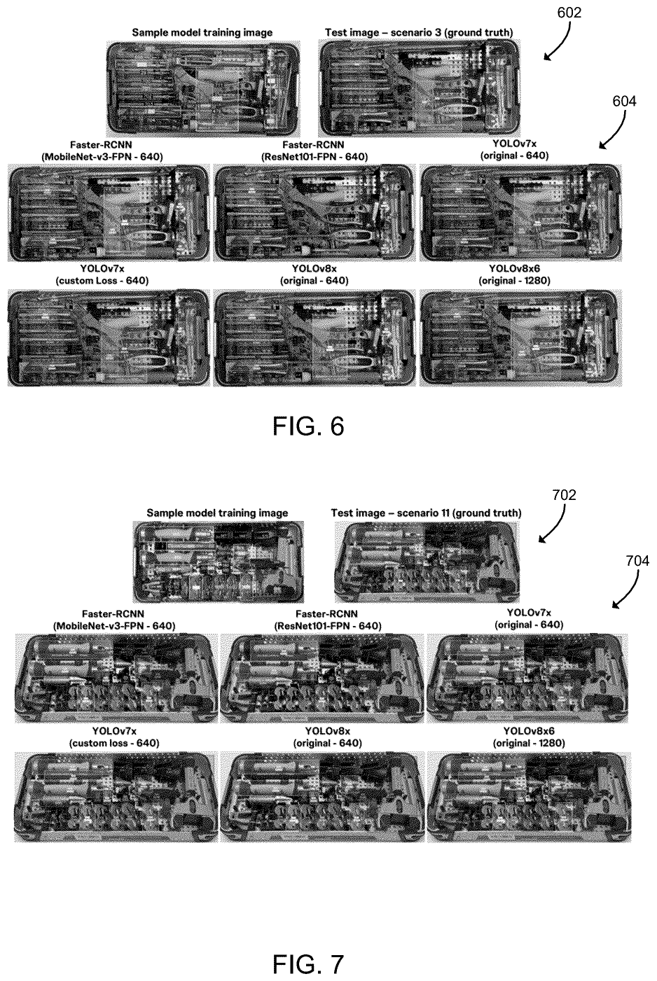

FIGS. 6 and 7 illustrate model predictions after applying a custom NMS to a sample test tray. In particular, FIGS. 6 and 7 show sample test images 602, 702 of two trays under scenarios 3 and 11, respectively, alongside the predictions 604, 704 generated by various models. As shown in FIG. 6, in scenario 3 where some components are missing from the tray, faster-RCNN models resulted in a significantly higher false negative rate under straight camera view angle compared to YOLO models. As shown in FIG. 7, under scenario 11, where the view angle deviates from straight, YOLO-based models significantly outperform Faster-RCNN based models when camera angle greatly deviates from the training data (straight view angle+/−5 degrees) and Faster-RCNN models fail to identify many components resulting in a high false negative rate.

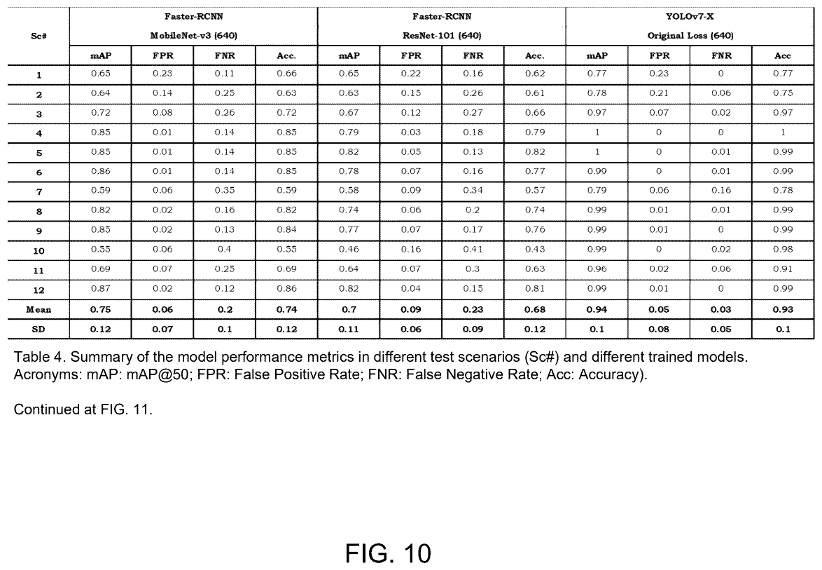

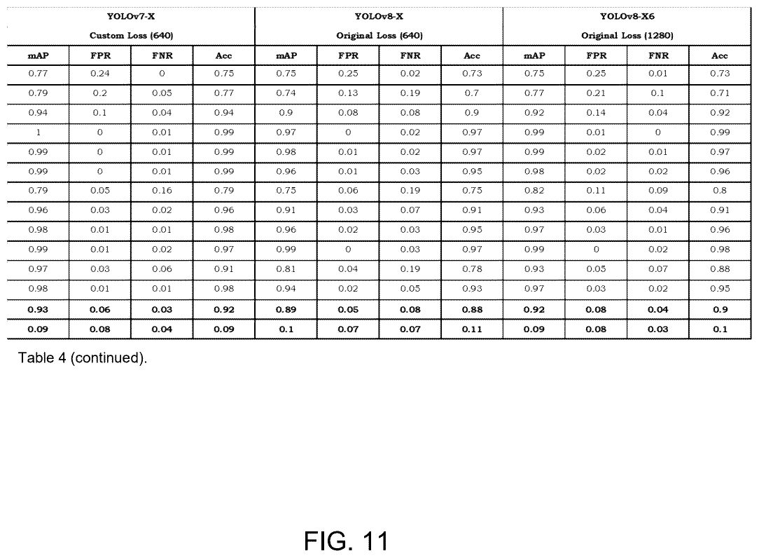

Table 4, shown in FIGS. 10-11, summarizes the performance metrics for all evaluated models. Overall, YOLO-based models outperformed Faster-RCNN models in every test scenario. In particular, YOLOv7-X with the original loss function resulted in the highest average performance metrics (mAP@50 of 0.94, false negative rate (FNR) of 2%, and false positive rate (FPR) of 6%). As shown, YOLO models exhibit superior robustness to variations in camera conditions such as height, view angle, and lighting compared to faster-RCNN architectures. The customization of the loss function did not apparently improve the YOLOv7-X model performance in the tests that were performed. In terms of detection accuracy, YOLOv7-X with custom loss function achieved the second highest performance (mAP=0.93), followed by YOLOv8-X6 (mAP=0.92), YOLOv8-X (mAP=0.89), faster-RCNN with MobileNet backbone (mAP=0.75), and finally faster-RCNN with ResNet-101 backbone (mAP=0.70).

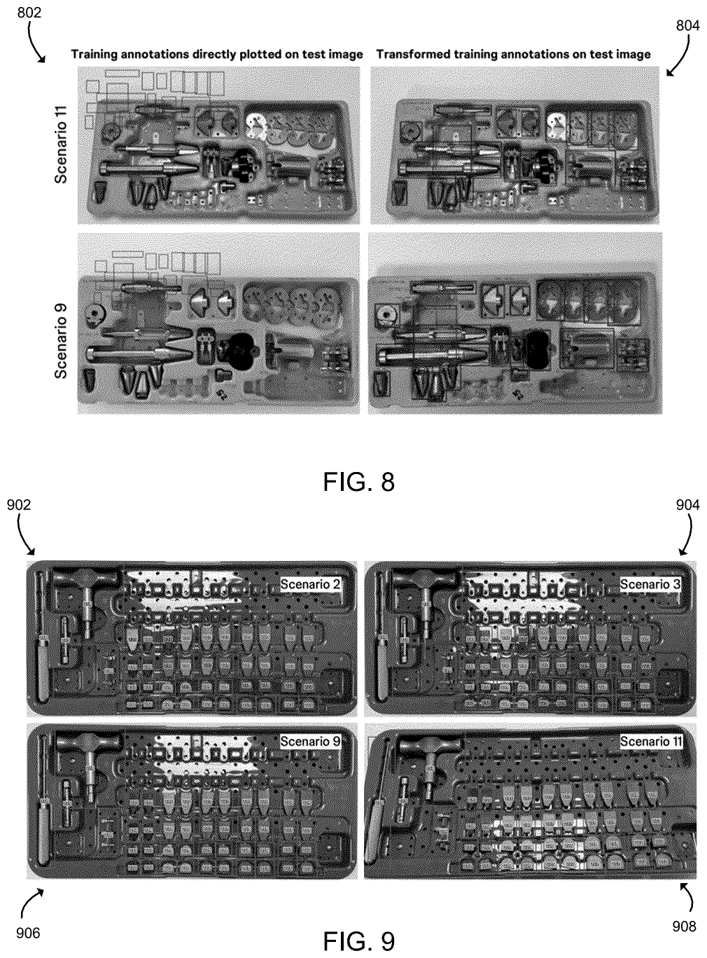

A pretrained LoFTR model was employed, followed by the USAC-MAGSAC method, and the annotations were seamlessly transformed from reference training images (first frame of the training videos) to the test images across all 12 scenarios with an impressive performance accuracy exceeding 99.5%. FIG. 8 illustrates an exemplary set of results from the developed layout verification pipeline demonstrating successful transformation of the reference annotations 802 to the test images 804 under two scenarios.

In particular, FIG. 8 includes a number of images 802, 804 showing example results of transforming training bounding boxes (red) on a test image using LOFTR+USAC-MAGSAC algorithms. The images 802 on the left show the direct representation of the annotations from training data displayed on test images without any modifications. The column 804 on the right shows the transformed training annotations (red). Blue boxes are the ground truth annotation on the test image.

FIG. 9 illustrates examples of the end-to-end inspection pipeline applied to a test tray 16 under different scenarios. All misplaced and missing components (shown in scenarios 2 and 3, images 902, 904) are correctly identified and flagged with a red bounding box displayed in their expected positions. Components that are detected with high confidence and their predicted positions match their designated slots are displayed with green bounding boxes (scenarios 9 and 11, images 906, 908). Green boxes indicate the components that are confidently detected by the object detector and their location matches their designated slot in the tray, and red boxes indicate the components that are missing (scenario 3, image 904) or misplaced from their designated slot (scenario 2, image 902).

Among all test scenarios, the three most challenging test scenarios across all models were 1, 2, and 7. Scenarios 1 and 2 involve the swapping or misplacement of similar-looking components within a tray. This difficulty can be attributed to several factors such as presence of component's mold/holders which secure them in place, overlapping components, and tray background details. Since all training images were captured with all components properly placed in their designated slots within each tray, consequently, in the training dataset, the bounding box for a component may encompass not only the component itself but also component holder pieces, tray background, or overlapping adjacent parts. The relatively lower model performance observed in scenario 7 was an expected outcome, as components appear drastically different when improperly placed (for instance placed upside down). The model has been trained to identify properly positioned component appearances, which explains the challenges encountered in this scenario.

### Discussion

In an illustrative embodiment, state-of-the-art computer vision techniques were implemented, and an end-to-end automated inspection system was developed to address the limitations of the current manual process. As shown in FIGS. 4-5, the developed system 10 executes a method 400 that utilizes an object detection model 206, a model prediction post-processing algorithm, followed by a tray layout verification algorithm that determines the presence and correct placement of each expected component in a particular tray. The single object detection model 206 was trained on a collection of 74 orthopedic surgical trays (with around 155,000 training/validation images). In the method 400 shown in FIGS. 4-5, the object detection model 206 is a trained YOLOv7-X with an original loss function.

After the object detection model 206 is executed, the method 400 advances to a model prediction post-processing algorithm at the block 410. The model prediction post-processing algorithm includes a customized NMS algorithm that filters the object detection predictions. It utilizes a custom configuration file 210 that is accessed based on the tray's unique identifier 26. The file 210 provides a list of component IDs belonging to each tray. In the first block 412 of the algorithm 410, the algorithm utilizes the tray configuration data and enforces a tray-specific expected component IDs constraint on model predictions to filter out the predicted classes that do not belong to the tray under inspection. In the next block 414, the algorithm applies a class-based NMS to detections such that for each expected class (component ID), only the prediction with the highest confidence score is maintained and the rest are removed from the predictions. In the remaining block 416 of algorithm, the algorithm applies an Intersection over Union (IoU) condition on top of the class-based NMS to keep the expected quantity of predictions that meet two criteria: they are non-overlapping (IoU<50%) and have the highest confidence scores among all predictions for the same class.

After the post-processing algorithm, the method 400 advances to the tray layout verification algorithm at block 418, which includes a block 420 in which key points from the training and test images are matched by a LoFTR model to co-register the two images. In the illustrative embodiment, the LoFTR model extracts the optimal matching keypoints between the first frame of the training video and test images. The tray layout verification algorithm then uses a novel variation of the RANSAC algorithm, USAC-MAGSAC, to process the predicted matching keypoints and calculate the transformation matrix from the train to test image. Given the transformation matrix, all ground truth bounding boxes from the reference train image are translated into the test image by the algorithm in the next block 422, providing the anticipated position of each component within the test image. The algorithm subsequently compares the center of the predicted bounding boxes with their corresponding center of the expected bounding boxes at block 424. A predicted bounding box is classified as “correct” and may be displayed with a “green” box if its centroid location is within 20% of the expected position. Conversely, if the predicted centroid location deviates from the expected position or if there is no prediction for an expected component, that component is marked as “missing/misplaced” and may be depicted with a red bounding box in the expected location.

Quality control and product integrity are paramount in supply chain and industrial processes. Manual inspections are costly, labor-intensive, and time-consuming. An automated visual inspection tool powered by computer vision ensures consistency and offers fast and reliable quality assessment. As disclosed herein, an end-to-end pipeline for intelligent visual inspection of surgical trays in the medical devices industry was implemented. The disclosed computer vision-based solution enabled an accurate, efficient, and scalable inspection process, overcoming challenges faced in the manual inspection of surgical trays. The disclosed pipeline includes enhanced YOLOv7 object detector, customized for its NMS function, and a custom layout verification process using LoFTR. A unique video dataset from 74 surgical trays, including 1039 unique classes, was collected to train a single object detection model 206, allowing seamless maintenance and scale-up. An overall mAP@0.5 of 0.94±0.10 and FPR at 0.05±0.08 as achieved across 12 realistic testing scenarios on a total of 139 inspection images.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An instrument tray inspection system, the system comprising:
a user interface adapter configured to (i) receive a test image of an instrument tray and (ii) determine a tray identifier, wherein the tray identifier is visually indicated on the instrument tray;
a recognition engine configured to generate a plurality of object predictions from the test image with a trained object recognition model, wherein each of the plurality of object predictions comprises a predicted location of a component within the instrument tray and a component identifier;
a recognition post-processor configured to post-process the plurality of object predictions with non-max suppression based on a predetermined tray configuration associated with the tray identifier, wherein the predetermined tray configuration comprises a plurality of expected component identifiers; and
a tray layout verifier configured to (i) determine whether the plurality of object predictions match a predetermined tray layout associated with the tray identifier, and (ii) clear the instrument tray for re-use in response to a determination that the plurality of object predictions match the predetermined tray layout associated with the tray identifier.

2. The system of claim 1, wherein the tray layout verifier is further configured to flag the instrument tray for further inspection in response to a determination that the plurality of object predictions do not match the predetermined tray layout associated with the tray identifier.

3. The system of claim 1, wherein the user interface adapter configured to receive the test image comprises the user interface adapter configured to receive the test image from a user device.

4. The system of claim 3, wherein the user interface adapter is further configured to transmit a user interface indicative of whether the plurality of object predictions match the predetermined tray layout associated with the tray identifier to the user device.

5. The system of claim 3, wherein the user interface adapter configured to determine the tray identifier comprises the user interface adapter configured to receive the tray identifier from the user device.

6. The system of claim 1, wherein the user interface adapter configured to determine the tray identifier comprises the user interface adapter configured to recognize the tray identifier in the test image.

7. The system of claim 1, wherein the recognition post-processor configured to post-process the plurality of object predictions comprises the recognition post-processor configured to filter object predictions based on the plurality of expected component identifiers of the predetermined tray configuration.

8. The system of claim 7, wherein the recognition post-processor configured to post-process the plurality of object predictions further comprises the recognition post-processor configured to perform non-max suppression based on the plurality of expected component identifiers in response to filtering of the object predictions.

9. The system of claim 8, wherein the predetermined tray configuration further comprises an expected quantity for each expected component identifier, and wherein the recognition post-processor configured to post-process the plurality of object predictions further comprises the recognition post-processor configured to remove any object predictions having an associated expected quantity value that is less than one.

10. The system of claim 8, wherein the recognition post-processor configured to post-process the plurality of object predictions further comprises the recognition post-processor configured to select the expected quantity of non-overlapping, highest-confidence object predictions for each expected component identifier having an associated expected quantity greater than one.

11. The system of claim 1, wherein:
the predetermined tray layout comprises a plurality of reference object identifications, wherein each reference object identification comprises an expected location of a component within the instrument tray and an expected component identifier; and
the tray layout verifier configured to determine whether the plurality of object predictions match the predetermined tray layout associated with the tray identifier comprises the tray layout verifier configured to:
register the test image and a reference image associated with the tray identifier to generate a transformation matrix;
transform the expected locations of the reference object identifications of the predetermined tray layout with the transformation matrix;
compare each reference object identification of the transformed predetermined tray layout to a corresponding object prediction of the plurality of object predictions; and
determine a presence indicator and a correct placement indicator for each reference object identification in response to a comparison of each reference object identification to the corresponding object prediction.

12. The system of claim 11, wherein the tray layout verifier configured to compare each reference object identification to the corresponding object prediction comprises the tray layout verifier configured to (i) determine whether an expected location of the reference object identification matches a predicted location of the corresponding object prediction within a predetermined threshold and (ii) determine whether an expected component identifier of the reference object identification matches a component identifier of the corresponding object prediction.

13. The system of claim 12, wherein the tray layout verifier configured to determine whether the expected location of the reference object identification matches the predicted location of the corresponding object prediction within the predetermined threshold comprises the tray layout verifier configured to determine whether a first centroid of a first bounding box of the expected location is within a predetermined percentage of a second centroid of a second bounding box of the predicted location.

14. A method for instrument tray inspection, the method comprising:
receiving, by a computing device, a test image of an instrument tray;
determining, by the computing device, a tray identifier, wherein the tray identifier is visually indicated on the instrument tray;
generating, by the computing device, a plurality of object predictions from the test image with a trained object recognition model, wherein each of the plurality of object predictions comprises a predicted location of a component within the instrument tray and a component identifier;
post-processing, by the computing device, the plurality of object predictions with non-max suppression based on a predetermined tray configuration associated with the tray identifier, wherein the predetermined tray configuration comprises a plurality of expected component identifiers;
determining, by the computing device, whether the plurality of object predictions match a predetermined tray layout associated with the tray identifier; and
clearing, by the computing device, the instrument tray for re-use in response to determining that the plurality of object predictions match the predetermined tray layout associated with the tray identifier.

15. The method of claim 14, wherein receiving the test image comprises receiving the test image from a user device, and wherein the method further comprising:
generating, by the user device, a user interface indicative of whether the plurality of object predictions match the predetermined tray layout associated with the tray identifier.

16. The method of claim 14, wherein post-processing the plurality of object predictions comprises filtering object predictions based on the plurality of expected component identifiers of the predetermined tray configuration.

17. The method of claim 14, wherein:
the predetermined tray layout comprises a plurality of reference object identifications, wherein each reference object identification comprises an expected location of a component within the instrument tray and an expected component identifier; and
determining whether the plurality of object predictions match the predetermined tray layout associated with the tray identifier comprises:
registering the test image and a reference image associated with the tray identifier to generate a transformation matrix;
transforming the expected locations of the reference object identifications of the predetermined tray layout with the transformation matrix;
comparing each reference object identification of the transformed predetermined tray layout to a corresponding object prediction of the plurality of object predictions; and
determining a presence indicator and a correct placement indicator for each reference object identification in response to comparing each reference object identification to the corresponding object prediction.

18. One or more non-transitory, computer readable storage media comprising a plurality of instructions that, in response to being executed, cause a computing device to:
receive a test image of an instrument tray;
determine a tray identifier, wherein the tray identifier is visually indicated on the instrument tray;
generate a plurality of object predictions from the test image with a trained object recognition model, wherein each of the plurality of object predictions comprises a predicted location of a component within the instrument tray and a component identifier;
post-process the plurality of object predictions with non-max suppression based on a predetermined tray configuration associated with the tray identifier, wherein the predetermined tray configuration comprises a plurality of expected component identifiers;
determine whether the plurality of object predictions match a predetermined tray layout associated with the tray identifier; and
clear the instrument tray for re-use in response to determining that the plurality of object predictions match the predetermined tray layout associated with the tray identifier.

19. The one or more non-transitory, computer readable storage media of claim 18, wherein to post-process the plurality of object predictions comprises to filter object predictions based on the plurality of expected component identifiers of the predetermined tray configuration.

20. The one or more non-transitory, computer readable storage media of claim 19, wherein to post-process the plurality of object predictions further comprises to:
perform non-max suppression based on component identifier in response to filtering the object predictions; and
select an expected quantity of non-overlapping, highest-confidence object predictions for each expected component identifier having an associated expected quantity greater than one, wherein the predetermined tray configuration further comprises the expected quantity for each expected component identifier.
